(19) United States
(12) Patent Application Publication
(10) Pub. No.: US 2007/0112233 A1
(43) Pub. Date: May 17, 2007

(54) APPARATUS FOR PREPARING VINYL CHLORIDE BY PYROLYSIS OF 1,2-DICHLOROETHANE AND METHOD OF PREPARING VINYL CHLORIDE USING THE SAME

(75) Inventors: Sung Won Kim, Daejeon-city (KR); Ye Hoon Kim, Daejeon-city (KR); DongHyun Jo, Daejeon-city (KR); Jongwook Bae, Daejeon-city (KR); Sang-Seung Oh, Cheonan-city, Chungcheongnam-do (KR); Soonyeel Lee, Daejeon-city (KR); Gimoon Nam, Seoul (KR)

Correspondence Address: CANTOR COLBURN, LLP, 55 GRIFFIN ROAD SOUTH, BLOOMFIELD, CT, 06002, US

(73) Assignee: LG CHEM, LTD., Seoul (KR)

(21) Appl. No.: 11/559,029

(22) Filed: Nov. 13, 2006

(30) Foreign Application Priority Data
Nov. 17, 2005 (KR) 10-2005-0110075

(51) Int. Cl. C07C 17/10

(52) U.S. Cl. 570/230; 422/187

(57) ABSTRACT

Provided are an apparatus for preparing vinyl chloride including: a pyrolysis reactor in which 1,2-dichloroethane and inert solid particles are mixed to generate vinyl chloride and hydrochloric acid; a first separator receiving the vinyl chloride, hydrochloric acid, and inert solid particles from the pyrolysis reactor and separating the vinyl chloride and hydrochloric acid from the inert solid particles; and a regeneration reactor receiving the separated inert solid particles from the first separator and regenerating the inert solid particles by burning the inert solid particles in a high temperature to remove coke deposited on the inert solid particles, wherein the restoration reactor is connected to the pyrolysis reactor to resupply the restored inert solid particles to the pyrolysis reactor, and a method of preparing vinyl chloride using the same. According to the apparatus and the method, the conversion rate can be improved, the productivity can be improved by effectively preventing interruption due to coke deposition, and the thermal efficiency can be enhanced by reusing thermal energy of sold particles heat treated at a high temperature in the regeneration reactor in the pyrolysis.

### CROSS-REFERENCE TO RELATED PATENT APPLICATION

This application claims the benefit of Korean Patent Application No. 10-2005-0110075, filed on Nov. 17, 2005, in the Korean Intellectual Property Office, the disclosure of which is incorporated herein in its entirety by reference.

### BACKGROUND OF THE INVENTION

1. Field of the Invention

The present invention relates to an apparatus for preparing vinyl chloride by pyrolysis of 1,2-dichloroethane and a method of preparing vinyl chloride using the same, and more particularly, to an apparatus for continuously preparing vinyl chloride without interruption of the reaction system by generating vinyl chloride using pyrolysis of 1,2-dichloroethane in a reactor and removing coke which is generated in the pyrolysis by depositing coke on solid particles and burning coke in a regeneration reactor.

2. Description of the Related Art

A method of preparing vinyl chloride by a gas phase pyrolysis of 1,2-dichloroethane is widely used in an industrial size and the process is disclosed in publications (Ulmann's Encyclopedia of Industrial Chemistry, 5th Edition, 1986, vol. 6, 287-289). Generally, a pyrolysis of 1,2-dichloroethane is performed in a tubular reactor at a temperature in the range of 400 to 550° C. for 10 to 20 seconds. The conversion rate is in the range of 50 to 60%, and the selectivity is in the range of 95 to 99%. In order to improve the conversion rate in the process, it is necessary to increase the temperature in the reactor and a residence time of the reactants. However, when the temperature is increased, a large amount of coke, a byproduct, is generated and the coke is deposited on the inside wall of the tubular reactor. Thus, the operation of the tubular reactor regularly stops to remove the deposited coke, and there are limits to improving the conversion rate by raising the temperature in the reactor.

In order to overcome such limitation, a method of diluting 1,2-dichloroethane using hydrochloric acid prior to the pyrolysis is disclosed in European Patent No. 195,719. However, such process is complex and the manufacturing costs increases. U.S. Pat. No. 5,488,190 discloses a method of improving the conversion rate and selectivity by mixing 1,2-dichloroethane with a high temperature gas or solid particles to increase the temperature to 500 to 750° C. during the reaction, staying the reactants for 0.01 to 0.25 seconds and rapidly cooling the reactor. A thermal medium such as the high temperature gas or solid particles was reported as a means to more rapidly increase the temperature of the tubular reactor compared to a conventional tubular reactor. However, the reaction system cannot be easily controlled since the residence time of the reactants is too short, and a possibility of coke which can be generated as a result of increasing conversion rate by raising the temperature of the reactor and a method of removing the generated coke are not described in the method.

### SUMMARY OF THE INVENTION

The present invention provides an apparatus for continuously preparing vinyl chloride without interruption of the reaction system by increasing a conversion rate in generating vinyl chloride using pyrolysis of 1,2-dichloroethane and effectively preventing coke deposition in a reactor.

The present invention also provides a method of preparing vinyl chloride by pyrolysis of 1,2-dichloroethane using the apparatus.

According to an aspect of the present invention, there is provided an apparatus for preparing vinyl chloride including:

a pyrolysis reactor in which 1,2-dichloroethane and inert solid particles are mixed to generate vinyl chloride and hydrochloric acid;

a first separator receiving the vinyl chloride, hydrochloric acid, and inert solid particles from the pyrolysis reactor and separating the vinyl chloride and hydrochloric acid from the inert solid particles; and

a restoration reactor receiving the separated inert solid particles from the first separator and restoring the inert solid particles by burning the inert solid particles in a high temperature to remove coke deposited on the inert solid particles,

wherein the restoration reactor is connected to the pyrolysis reactor to resupply the restored inert solid particles to the pyrolysis reactor.

According to another aspect of the present invention, there is provided a method of preparing vinyl chloride including:

a) mixing the 1,2-dichloroethane with inert solid particles in a pyrolysis reactor to generate vinyl chloride and hydrochloric acid;

b) separating the generated vinyl chloride and hydrochloric acid from the inert solid particles;

c) removing coke deposited on the inert solid particles by burning the separated inert solid particles in a restoration reactor at a high temperature; and

d) recirculating the coke-removed inert solid particles into the pyrolysis reactor.

### BRIEF DESCRIPTION OF THE DRAWINGS

The above and other features and advantages of the present invention will become more apparent by describing in detail exemplary embodiments thereof with reference to the attached drawing in which:

FIG. 1 is a schematic diagram illustrating an apparatus for preparing vinyl chloride according to an embodiment of the present invention.

### DETAILED DESCRIPTION OF THE INVENTION

Hereinafter, the present invention will now be described more fully with reference to the accompanying drawing, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art.

The inventors of the present invention has found that a high conversion rate in a preparation of vinyl chloride by pyrolysis of 1,2-dichloroethane can be obtained and an interruption of reaction system due to coke generation in a reactor can be effectively prevented using a fluidization or fluidized bed technique. The fluidization or fluidized bed technique is a technique that converts solid particles to have a liquid-like characteristic by flowing a medium such as gas or liquid on a solid particle layer, and is used in a process of using solid particles. Particularly, a circulating fluidized bed technique, which is one field of fluidized bed techniques, is a technique that performs a reaction at a high gas flow velocity that can float and transfer all of the solid particles, and represents a high mixing efficiency and heat transfer efficiency (Fluidizing Engineering, 2^{nd} Edition, 1991, 359-395).

The present invention applies the circulating fluidized bed technique to the preparation of vinyl chloride by pyrolysis of 1,2-dichloroethane. According to the apparatus and the method of an embodiment of the present invention, the conversion rate of 1,2-dichloroethane can be noticeably increased by pyrolysis at a high temperature, productivity can be increased by a continuous operation in the reaction system without interruption due to coke removing, and thermal efficiency can be increased by applying thermal energy of solid particles heat treated in the regeneration stage to the pyrolysis.

Hereinafter, the present invention will be described in further detail with reference to the accompanying drawings.

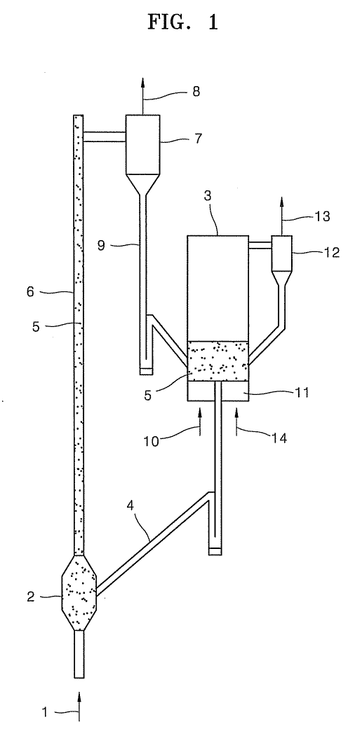

FIG. 1 is a schematic diagram illustrating an apparatus for preparing vinyl chloride according to an embodiment of the present invention. The apparatus for preparing vinyl chloride includes: a pyrolysis reactor 6 in which 1 ,2-dichloroethane and inert solid particles 5 are mixed to generate vinyl chloride and hydrochloric acid; a first separator 7 receiving the vinyl chloride, hydrochloric acid, and inert solid particles from the pyrolysis reactor 6 and separating the vinyl chloride and hydrochloric acid from the inert solid particles; and a restoration reactor 3 receiving the separated inert solid particles 5 from the first separator 7 and burning the inert solid particles in a high temperature to remove coke deposited on the inert solid particles, wherein the restoration reactor 3 is connected to the pyrolysis reactor 6 to resupply the restored inert solid particles to the pyrolysis reactor 6.

That is, the pyrolysis reactor 6, the first separator 7 and the restoration reactor 3 are connected in this order, and the restoration reactor 3 and the pyrolysis reactor 6 are connected to each other in a circular structure, and thus the solid particles 5 which are restored in the restoration reactor 3 can be resupplied into the pyrolysis reactor 6. Thus, such structure is economically effective and prevents interruptions due to the solid particles 5.

The pyrolysis reactor 6 may be a tubular reactor or a rectangular reactor, and the cross-section of the pyrolysis reactor 6 may be a circle, a triangle, a rectangle, a pentagon, a hexagon, or any shape having an obtuse angle.

In addition, the inert solid particles 5 included in the apparatus for preparing vinyl chloride may be any inert solid particles, the thermal energy of which can be used in the pyrolysis of 1,2-dichloroethane and which are heated at a high temperature. In particular, the solid particle may be silica, alumina, and silica alumina, or a composition thereof.

An average particle size of the inert solid particles 5 may be in the range of 5 to 1,000 μm, and more preferably in the range of 20 to 300 μm. When the average particle size of the inert solid particles 5 is less than 5 μm, inert solid particles agglomerate, and thus floating and flowing properties of the inert solid particles become poor and the obtained vinyl chloride cannot be easily separated from the inert solid particles. When the average particle size of the inert solid particles 5 is greater than 1,000 μm, the inert solid particles cannot be easily fluidized in the pyrolysis 6 and transferred to an upper portion of the pyrolysis reactor 6 although the obtained vinyl chloride can be easily separated from the inert solid particles.

In the pyrolysis reactor 6, the inert solid particles 5 and 1,2-dichloroethane as a raw material supplied through a reactant inlet 1 are mixed in a mixing chamber 2. The mixture is uniformly mixed by flowing in the pyrolysis reactor 6, and thus pyrolysis of 1,2-dichloroethane occurs. In other words, the inert solid particles 5 and the raw material are primarily mixed in the mixing chamber 2illustrated in FIG. 1, and the mixture is uniformly mixed by flowing in the pyrolysis reactor 6.

The vinyl chloride and hydrochloric acid which are products of the pyrolysis of 1,2-dichloroethane in the pyrolysis reactor 6 are transferred to a first separator 7 through the flow in the apparatus.

The vinyl chloride and hydrochloric acid are separated from the inert solid particles 5 using a cyclone or a similar gas/solid separator in the first separator 7, and the separated vinyl chloride and hydrochloric acid are discharged through a generated gas outlet 8.

The separated inert solid particles 5 are transferred from the first separator 7 to a restoration reactor 3 through the flow in the apparatus and a solid particle transferring tube 9. The transferred inert solid particles 5 are restored by removing coke deposited on the solid particles through burning coke using air injected through an air inlet 10 and methane injected through a methane inlet 14.

The restored solid particles obtained for the restoration reactor 3 are transferred to the mixing chamber 2 through a solid particle inlet 4 and mixed with 1,2-dichloroethane, and thus resupplied to the pyrolysis reactor 6. Waste gases such as carbon dioxide and carbon monoxide produced by the coke combustion in the restoration reactor 3 are separated from the scattered solid particles and discharged through a waste gas outlet 13. The separated solid particles in a second separator 12 are collected and transferred to the restoration reactor 3.

The apparatus for preparing vinyl chloride according to an embodiment of the present invention may further include a device separating vinyl chloride by cooling the vinyl chloride and hydrochloric acid which are separated in the first separator 7. Pure vinyl chloride can be obtained using such device, and the obtained pure vinyl chloride can be used in a PVC manufacturing.

In the apparatus for preparing vinyl chloride, gaseous components including vinyl chloride and hydrochloric acid which are generated in the pyrolysis reactor 6 may not be contact with waste gases such as carbon dioxide and carbon monoxide which are generated in the restoration reactor 3 in the process of separating the generated gases from the inert solid particles 5 in the first separator 7. The apparatus for preparing vinyl chloride may further include the solid particle inlet 4 to prevent gaseous components generated in the restoration reactor 3 from being contact with gaseous components generated in the pyrolysis reactor 6, wherein the restoration reactor 3 is connected to the pyrolysis reactor 6.

Hereinafter, a method of preparing vinyl chloride by pyrolysis of 1,2-dichloroethane will be described in detail.

A method of preparing vinyl chloride according to an embodiment of the present invention includes a) mixing the 1,2-dichloroethane 1 with inert solid particles 5 in a pyrolysis reactor 6 to generate vinyl chloride and hydrochloric acid; b) separating the generated vinyl chloride and hydrochloric acid from the inert solid particles; c) removing coke deposited on the inert solid particles 5 by burning the separated inert solid particles 5 in a restoration reactor 3 at a high temperature; and d) recirculating the coke-removed inert solid particles 5 into the pyrolysis reactor 6.

In operation a), the pyrolysis reactor 6 may be a tubular reactor, and the cross-section of the pyrolysis reactor 6 may be a circle, a triangle, a rectangle, a pentagon, a hexagon, or any shape having an obtuse angle. The pyrolysis is performed in the tubular pyrolysis reactor 6 while 1,2-dichloroethane and inert solid particles 5 flow at a high velocity. Any tubular reactor which is commonly used in the art may be used as the tubular pyrolysis reactor 6 and the length and width thereof are not limited.

In addition, the inert solid particles 5 used in operation a) may be any inert solid particles, the thermal energy of which can be used in the pyrolysis of 1,2-dichloroethane, and may be heated at a high temperature. In particular, the inert solid particle 5 may be silica, alumina, and silica alumina, or a composition thereof.

The pyrolysis of 1,2-dichloroethane initiates at the temperature higher than 400° C., and thus the inside temperature of the pyrolysis reactor 6 may be maintained at 400° C. or higher. The inside temperature of the pyrolysis reactor 6 may be in the range of 400 to 1,000° C., and more preferably 450 to 700° C. When the inside temperature is less than 400° C., the pyrolysis efficiency may decrease and the conversion rate is too low. When the inside temperature is greater than 1,000° C., the amount of generated coke is too large, side reactions excessively occur, and the yield of vinyl chloride may decrease.

The length of residence time of the inert solid particles 5 in the pyrolysis reactor 6 is in inverse proportion to a velocity of reactant gases, and is influenced by the conversion rate and the amount of generated coke. The inert solid particles 5 may stay in the pyrolysis reactor 6 for 0.5 to 5 seconds, and more preferably 0.5 to 3 seconds. When the inert solid particles 5 stay for less than 0.5 seconds, the pyrolysis is not sufficiently performed, and thus the conversion rate is too low. On the other hand, when the inert solid particles 5 stay for longer than 5 seconds, the pyrolysis is excessively performed and a side reaction generating ethylene excessively occur, and thus the yield of vinyl chloride may decrease.

Pure 1,2-dichloroethane may only be used in the pyrolysis reactor 6, or inert solid particles such as nitrogen, argon, neon, or a mixture thereof may be used with 1,2-dichloroethane in the pyrolysis reactor 6.

Products of the pyrolysis discharged from the pyrolysis reactor 6 and the inert solid particles 5 on which coke are deposited may be separated in the first separator 7. A cyclone or a similar gas/solid separator may be used.

The inert solid particles 5 transferred to the restoration reactor 3 is burned at a high temperature using oxygen or air or a mixture of combustible gas and oxygen or air in the restoration reactor 3. Coke is burned and removed from the inert solid particles 5 as carbon dioxide, carbon monoxide, etc. The combustion method may be a fluidized bed technique in which combustion is performed while particles float, but is not limited thereto. In the fluidized bed technique, the inert solid particles 5 may be scattered and disposed in an upper portion of the restoration reactor 3 with the generated carbon dioxide and nitrogen, etc. However, the inert solid particles 5 may be collected in the second separator 12 connected to the restoration reactor 3 and resupplied to the restoration reactor 3, and waste gases are discharged through the waste gas outlet 13.

The inert solid particles 5 in which coke is removed in the restoration reactor 3 are resupplied to the pyrolysis reactor 6 through the solid particle inlet 4 and reused. The solid particle inlet 4 is installed to prevent gaseous components generated in the restoration reactor 3 from being contact with gaseous components generated in the pyrolysis reactor 6. A partial or entire thermal energy of the inert solid particles 5 heat treated in the restoration reactor 3 may be used in the pyrolysis of 1,2-dichloroethane in the pyrolysis reactor 6 since the inert solid particles 5 resupplied to the pyrolysis reactor 6 is heat treated at a high temperature in the restoration reactor 3. Thus, the pyrolysis reactor 6 can be less heated by an additional heating device or the pyrolysis reactor 6 is not necessary to be heated for the pyrolysis.

Upon using the apparatus and method preparing vinyl chloride by pyrolysis of 1,2-dichloroethane according to an embodiment of the present invention, the conversion rate can be improved, the productivity can be improved by decreasing interruptions of reaction system to remove coke, which is a byproduct and generally deposited on the inside wall of the reactor, by attaching the coke to the solid particles and removing the coke through burning. In addition, the thermal efficiency can be improved by reusing the thermal energy of the inert solid particles 5 heat treated in the restoration reactor 3 in the pyrolysis reactor 6, and thus the pyrolysis reactor 6 can be less heated by an additional heating device or the pyrolysis reactor 6 is not necessary to be heated for the pyrolysis.

Hereinafter, the present invention will be described in further detail with reference to the following examples. These examples are for illustrative purposes only and are not intended to limit the scope of the present invention.

### EXAMPLE 1

1,2-dichloroethane pre-heated at 260° C. was supplied to a mixing chamber 2, and mixed with high temperature silica sand 5 (Kanto Chem.) having an average diameter of 120 to 230μm which was supplied from a regeneration reactor 3. The temperature of the bottom portion of a pyrolysis reactor 6 was 600° C., and pyrolysis of 1,2-dichloroethane was initiated while the mixture flowed to an upper portion in the pyrolysis reactor 6. The amount of the 1,2-dichloroethane 1 supplied to the pyrolysis reactor 6 was 5.82 g/min, the amount of circulated silica particles 5 was 22.9 g/min, and the velocity of 1,2-dichloroethane in the pyrolysis reactor 6 was 2.16 m/s. The inside temperature in the vicinity of outlet of the pyrolysis reactor 6 was 550° C. The generated gases, unreacted gases, and silica particles 5 on which coke is deposited were discharged to a first separator 7 connected to the outlet of the pyrolysis reactor 6. The gases and silica particles were separated using a cyclone, and the gases and silica particles were released to outside of the pyrolysis reactor 6, and then cooled down and separated. Thus, pure vinyl chloride was obtained. The silica particles 5 on which coke was deposited were supplied to the regeneration reactor 3 through a solid particle transferring tube 9 which was connected to the regeneration reactor 3. Nitrogen was injected in the solid particle transferring tube 9 to prevent the generated gases in the pyrolysis reactor 6 from flowing into the regeneration reactor 3 while the silica particles 5 were transferred. Methane gas was injected through a methane inlet 14 with the velocity of 4.42 g/min, and air was injected through an air inlet 10 with the velocity of 73.62 g/min. Then, the injected air, methane, and silica particles 5 were floated in the restoration reactor 3 through an air distributor 11, and the regeneration reactor 3 was heated. The inside temperature of the heated regeneration reactor 3 was 740° C. Waste gases generated by burning coke in the regeneration reactor 3 and scattered minute particles were removed in a second separator 12 and discharged through a waste gas outlet 13. Coke-removed high temperature silica particles 5 were resupplied to the pyrolysis reactor 6 while nitrogen was injected into the solid particle inlet 4. The process was repeated.

### EXAMPLE 2

Vinyl chloride was prepared in the same manner as in Example 1 except that the inside temperature of the pyrolysis reactor 6 was 615° C., and the amount of circulated silica particles 5 was 25.7 g/s.

### EXANPLE 3

Vinyl chloride was prepared in the same manner as in Example 1 except that the inside temperature of the pyrolysis reactor 6 was 650° C.

### COMPARATIVE EXAMPLE 1

A pyrolysis of 1,2-dichloroethane was performed in a conventional furnace, i.e., a tubular reactor using a known method (Ulmann's Encyclopedia of Industrial Chemistry, 5th Edition, 1986, vol. 6, 287-289) at 490° C., wherein solid particles were not added and a regeneration reactor was not installed.

The products obtained in Examples 1 to 3 and Comparative Example 1 were cooled and separated, and the amount was analyzed. The conversion rate was calculated using Formula 1 below.
Conversion rate=(weight of injected 1,2-dichloroethane−weight of unreacted 1,2-dichloroethane)/(weight of injected 1,2-dichloroethane)×100 Formula 1

The results are shown in Table 1.

TABLE 1

| | | | | Comparative |
|---|---|---|---|---|
| | Example 1 | Example 2 | Example 3 | Example |
| Reaction temperature (° C.) | 600 | 615 | 650 | 490 |
| Injected EDC amount (g/min) | 5.82 | 5.82 | 5.82 | 1.00 |
| Staying time of EDC (sec) | 2 | 2 | 2 | 18 |
| Solid particle | silica | silica | silica | none |
| Solid particle circulating | 22.9 | 25.7 | 22.9 | |
| amount (g/s) | | | | |
| Restoration temperature (° C.) | 740 | 740 | 740 | |
| Injected air amount (g/min) | 73.62 | 70.26 | 73.62 | |
| Injected methane amount (g/min) | 4.42 | 4.22 | 4.42 | |
| Conversion rate | 98.4 | 99.6 | 99.8 | 56.5 |
| (weight (g/min)/mole fraction) | | | | |
| vinyl chloride | 2.840/0.390 | 2.609/0.356 | 2.401/0.335 | 0.334/0.340 |
| EDC | 0.093/0.008 | 0.023/0.002 | 0.011/0.001 | 0.435/0.280 |
| Hydrogen chloride | 2.290/0.538 | 2.380/0.558 | 2.426/0.570 | 0.201/0.350 |
| coke | | | | 0.001/0.019 |
| byproduct | 0.586/0.061 | 0.793/0.083 | 0.730/0.076 | 0.029/0.001 |
| (coke amount removed in | 0.011/0.003 | 0.015/0.001 | 0.066/0.018 | |
| the regeneration reactor | | | | |
| (g/min)/mole fraction) | | | | |

EDC = 1,2-dichloroethane

As shown in Table 1, the conversion rate of the apparatus for preparing vinyl chloride by pyrolysis of 1,2-dichloroethane according to the present invention was greater than 98%, which is noticeably higher than that of conventional apparatuses having 50 to 60%.

In addition, the pyrolysis can be smoothly performed at a high temperature of 600° C. or higher without interruption of reaction system by removing coke through burning.

Upon using the apparatus and method of circulating solid particles in the reaction system in preparing vinyl chloride by pyrolysis of 1,2-dichloroethane, the conversion rate can be noticeably improved, the amount of coke which was a byproduct of the pyrolysis and generally deposited on the inside wall of the reactor can be decreased by attaching the coke to the solid particles and removing the coke through burning, and thermal efficiency can be improved by circulating the solid particles heat treated at a high temperature in the regeneration reactor into the reactor and reusing the thermal energy.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An apparatus for preparing vinyl chloride comprising:
a pyrolysis reactor in which 1,2-dichloroethane and inert solid particles are mixed to generate vinyl chloride and hydrochloric acid;
a first separator receiving the vinyl chloride, hydrochloric acid, and inert solid particles from the pyrolysis reactor and separating the vinyl chloride and hydrochloric acid from the inert solid particles; and
a regeneration reactor receiving the separated inert solid particles from the first separator and regenerating the inert solid particles by burning the inert solid particles in a high temperature to remove coke deposited on the inert solid particles,
wherein the regeneration reactor is connected to the pyrolysis reactor to resupply the restored inert solid particles to the pyrolysis reactor.

2. The apparatus of claim 1, wherein the pyrolysis reactor is a tubular reactor or a rectangular reactor.

3. The apparatus of claim 1, wherein the inert solid particle is selected from the group consisting of silica, alumina, and silica alumina.

4. The apparatus of claim 1, wherein the average particle size of the inert solid particles is in the range of 5 to 1,000 μm.

5. The apparatus of claim 1, wherein the separated vinyl chloride and hydrochloric acid are cooled in the first separator and the apparatus further comprises a second separator separating the vinyl chloride from the hydrochloric acid.

6. The apparatus of claim 1, further comprising a solid particle inlet to prevent gaseous components generated in the regeneration reactor from being contact with gaseous components generated in the pyrolysis reactor, wherein the regeneration reactor is connected to the pyrolysis reactor.

7. A method of preparing vinyl chloride comprising:
a) mixing the 1,2-dichloroethane with inert solid particles in a pyrolysis reactor to generate vinyl chloride and hydrochloric acid;
b) separating the generated vinyl chloride and hydrochloric acid from the inert solid particles;
c) removing coke deposited on the inert solid particles by burning the separated inert solid particles in a regeneration reactor at a high temperature; and
d) recirculating the coke-removed inert solid particles into the pyrolysis reactor.

8. The method of claim 7, wherein the temperature in the pyrolysis reactor is in the range of 400 to 1000° C.

9. The method of claim 7, wherein the 1,2-dichloroethane and the inert solid particles stay in the pyrolysis reactor for 0.5 to 5 seconds.

10. The method of claim 7, wherein at least one inert gas selected from the group consisting of nitrogen, argon, neon, and helium is injected into the pyrolysis reactor.

11. The method of claim 7, wherein the temperature in the restoration reactor is in the range of 500 to 1000° C.

12. The method of claim 7, wherein partial or entire thermal energy of the inert solid particles heat treated in the regeneration reactor is used in the pyrolysis of 1,2-dichloroethane in the pyrolysis reactor.

13. The method of claim 7, further comprising cooling the separated vinyl chloride and hydrochloric acid, and separating the vinyl chloride from the hydrochloric acid to obtain vinyl chloride.
